(19) United States
(12) Patent Application Publication
(10) Pub. No.: US 2018/0092646 A1
(43) Pub. Date: Apr. 5, 2018

(54) Skin Tag Removal Assembly

(71) Applicant: Gene Copenspire, Federal Way, WA (US)

(72) Inventor: Gene Copenspire, Federal Way, WA (US)

(21) Appl. No.: 15/285,981

(22) Filed: Oct. 5, 2016

(51) Int. Cl. A61B 17/12

(52) U.S. Cl. CPC A61B 17/12009; A61B 2017/00761

(57) ABSTRACT

A skin tag removal assembly for non surgical remove of skin tags includes a pair of straws. A string is extended through each of the straws. The string is tied into a loop and the loop is selectively positioned around a skin tag. The loop is selectively tightened to restrict blood circulation to the skin tag. Thus, the skin tag is non-surgically removed.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to removal devices and more particularly pertains to a new removal device for non surgical removal of skin tags.

### BRIEF SUMMARY OF THE INVENTION

An embodiment of the disclosure meets the needs presented above by generally comprising a pair of straws. A string is extended through each of the straws. The string is tied into a loop and the loop is selectively positioned around a skin tag. The loop is selectively tightened to restrict blood circulation to the skin tag. Thus, the skin tag is non-surgically removed.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a skin tag removal assembly according to an embodiment of the disclosure.

FIG. 2 is a front phantom view of an embodiment of the disclosure.

FIG. 3 is a cross sectional view taken along line 3-3 of FIG. 2 of an embodiment of the disclosure.

FIG. 4 is a perspective in-use view of an embodiment of the disclosure.

### DETAILED DESCRIPTION OF THE INVENTION

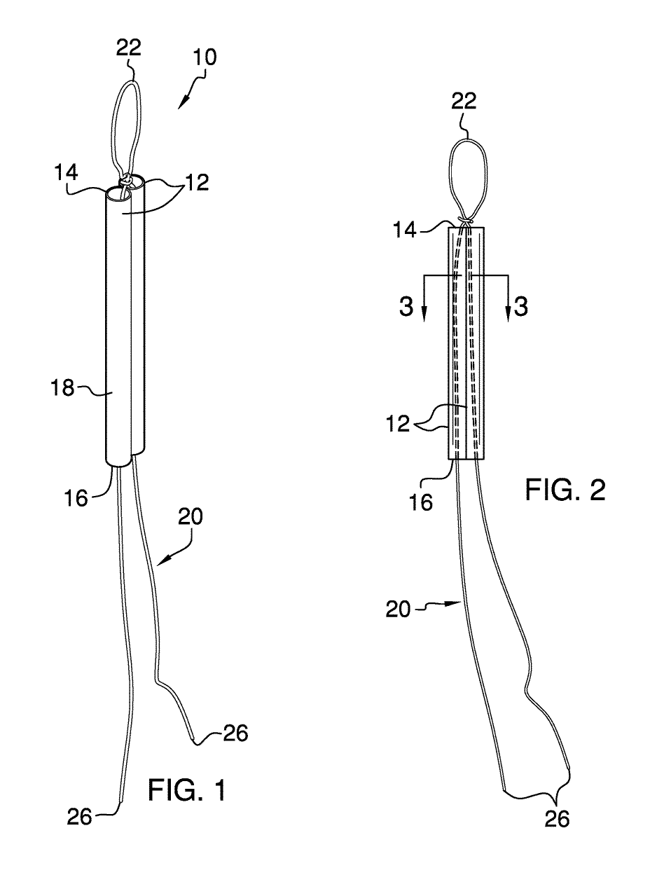

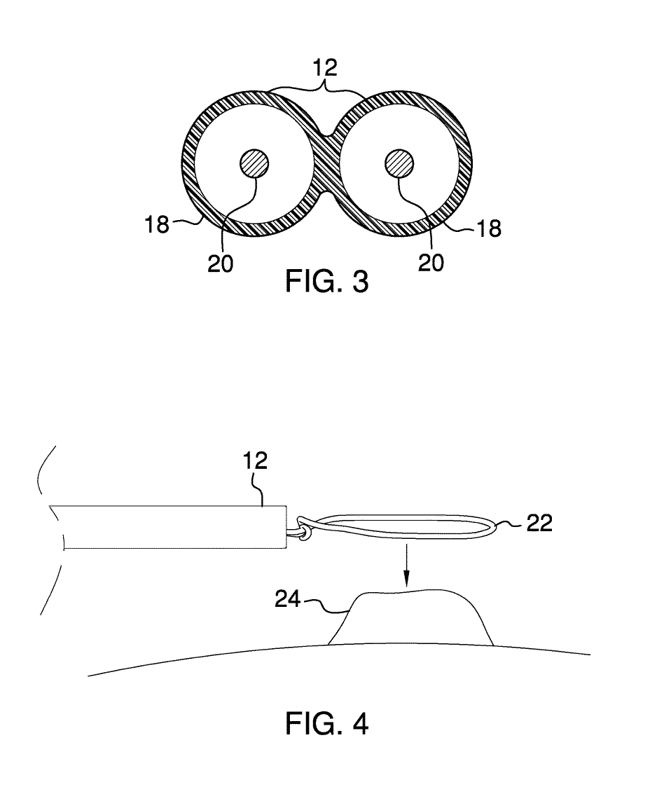

With reference now to the drawings, and in particular to FIGS. 1 through 4 thereof, a new removal device embodying the principles and concepts of an embodiment of the disclosure and generally designated by the reference numeral 10 will be described.

As best illustrated in FIGS. 1 through 4, the skin tag removal assembly 10 generally comprises a pair of straws 12. Each of the straws 12 has a first end 14, a second end 16 and an outer wall 18. The outer wall 18 corresponding to each of the straws 12 is coupled together. Thus, the straws 12 are coextensive with each other.

A string 20 is extended through each of the straws 12. The string 20 is tied into a loop 22 and the loop 22 is selectively positioned around a skin tag 24. The loop 22 may be an overhand knot or the like. The loop 22 is selectively tightened thereby restricting blood circulation to the skin tag 24. Thus, the skin tag 24 is removed non-surgically. The string 20 may be dental floss or the like.

The string 20 has pair of loose ends 26. The string 20 extends outwardly from the second end 16 corresponding to each of the straws 12. Each of the loose ends 26 is spaced from the corresponding second end 16. The string 20 may extend outwardly from the straws 12 a distance ranging between ten cm and fifteen cm.

The string 20 extends outwardly from the first end 14 corresponding to each of the straws 12. The loop 22 is spaced from the corresponding first end 14. Each of the loose ends 26 is selectively urged away from the corresponding second end 16. Thus, a diameter of the loop 22 is selectively reduced.

In use, the straws 12 are manipulated to position the loop 22 around a skin tag 24. The string 20 is drawn outwardly from the second end 16 corresponding to each straw. Thus, the loop 22 is tightened around the skin tag 24. The loop 22 restricts blood flow to the skin tag 24. The string 20 is tied off below the loop 22 and the string 20 is cut off. Thus, the loop 22 remains tightened around the skin tag 24. The skin tag 24 falls off after a duration of time due to loss of circulation.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A skin tag removal assembly being configured to facilitate non-surgical removal of skin tags, said assembly comprising:
a pair of straws; and
a string being extended through each of said straws, said string being tied into a loop wherein said loop is configured to be positioned around a skin tag, said loop being selectively tightened wherein said loop is configured to restrict blood circulation to the skin tag thereby facilitating non-surgical removal of the skin tag.

2. The assembly according to claim 1, wherein each of said straws has a first end, a second end and an outer wall, said outer wall corresponding to each of said straws being coupled together such that said straws are coextensive with each other.

3. The assembly according to claim 1, wherein:
each of said straws has a second end; and
said string has pair of loose ends, said string extending outwardly from said second end corresponding to each of said straws having each of said loose ends being spaced from said corresponding second end.

4. The assembly according to claim 3, wherein:
each of said straws has a first end; and
said string extends outwardly from said first end corresponding to each of said straws having said loop being spaced from said corresponding first end, each of said loose ends being urged away from said corresponding second end such that a diameter of said loop is selectively reduced.

5. A skin tag removal assembly being configured to facilitate non-surgical removal of skin tags, said assembly comprising:
a pair of straws, each of said straws having a first end, a second end and an outer wall, said outer wall corresponding to each of said straws being coupled together such that said straws are coextensive with each other; and
a string being extended through each of said straws, said string being tied into a loop wherein said loop is configured to be positioned around a skin tag, said loop being selectively tightened wherein said loop is configured to restrict blood circulation to the skin tag thereby facilitating non-surgical removal of the skin tag, said string having pair of loose ends, said string extending outwardly from said second end corresponding to each of said straws having each of said loose ends being spaced from said corresponding second end, said string extending outwardly from said first end corresponding to each of said straws having said loop being spaced from said corresponding first end, each of said loose ends being urged away from said corresponding second end such that a diameter of said loop is selectively reduced.
